(19) United States
(12) Patent Application Publication
(10) Pub. No.: US 2026/0305665 A1
(43) Pub. Date: Oct. 8, 2026

(54) SALTY TASTE ENHANCER FOR FOOD/BEVERAGE

(71) Applicant: AJINOMOTO CO., INC., Tokyo (JP)

(72) Inventors: Takumi FUNAMIZU, Kanagawa (JP); Ryo MATSUMOTO, Kanagawa (JP)

(73) Assignee: AJINOMOTO CO., INC., Tokyo (JP)

(21) Appl. No.: 19/680,018

(22) Filed: May 18, 2026

Related U.S. Application Data
(63) Continuation of application No. PCT/JP2024/040947, filed on Nov. 19, 2024.

(30) Foreign Application Priority Data
Nov. 20, 2023 (JP) 2023-196656

(51) Int. Cl. A23L 27/21; A23L 2/56; A61N 1/36

(52) U.S. Cl. CPC A23L 27/21; A23L 2/56; A61N 1/36

(57) ABSTRACT

The present invention provides an enhancer of a salty taste of a food or beverage, that is consumed in a state under electrical stimulation, at the time of consuming the food or beverage, the enhancer containing a hydroxyl group-containing amino acid or basic amino acid, or a salt thereof, a food or beverage containing a hydroxyl group-containing amino acid or basic amino acid, or a salt thereof, which is consumed in a state under electrical stimulation, and the like.

### CROSS-REFERENCE TO RELATED APPLICATIONS

The present application is a Continuation of PCT/JP2024/040947, filed Nov. 19, 2024, which claims priority to JP 2023-196656, filed Nov. 20, 2023, the entire contents of which are incorporated herein by reference.

### TECHNICAL FIELD

The present invention relates to an enhancer of a salty taste at the time of consuming food or beverage, food or beverage whose salty taste at the time of consuming is enhanced, and the like.

### BACKGROUND ART

While sodium chloride is widely used as a basic seasoning for flavoring food, it is known that excessive sodium chloride intake has adverse effects on hypertension, cardiovascular diseases such as heart disease and the like, kidney disease, and the like.

In this connection, Patent Literature 1 discloses a method for enhancing the salty taste of salt-containing foods and beverages, characterized by the addition of an amino acid or a salt thereof and a metal salt. However, in the area of basic seasonings and foods, the amount of salt that can be reduced through combinations of formulations, without impairing intensity of salty taste or feeling of satisfaction, is about half.

Furthermore, a salty taste-enhancing effect by electrical stimulation is known. For example, Patent Literature 2 discloses a taste electrical stimulation device that adjusts taste, including enhancing salty taste by electrical stimulation. Non Patent Literature 1 discloses a higher salty taste-enhancing effect than conventional stimulation waveforms, which is achieved by combining the effects of presenting and stopping cathode stimulation and presenting anode stimulation. Non Patent Literature 2 discloses that anodic galvanic taste stimulation (GTS) is effective in enhancing the salty taste of NaCl aqueous solution.

On the other hand, Japanese people have a dietary habit with a high average salt intake of 10.1 g (National Health and Nutrition Survey, 2019), and patients with chronic kidney disease, in particular, are subject to a stricter salt intake standard of 3-6 g/day than for the general population. Therefore, reducing salt content beyond that of existing low-sodium foods is an issue.

### CITATION LIST

### Patent Literature

### [Patent Literature 1]

JP 2019-62778 A

### [Patent Literature 2]

JP 2018-42991 A

### Non Patent Literature

### [Non Patent Literature 1]

Frontiers in Virtual Reality, 2022, Vol. 3, p. 1-10

### [Non Patent Literature 2]

Journal of Robotics and Mechatronics, 2021, 33(5), p. 1128-1134

### SUMMARY OF INVENTION

### Technical Problem

The present invention has been made in view of the aforementioned circumstances, and aims to provide a salty taste enhancer that can more effectively enhance the salty taste at the time of consuming food or beverage that is consumed in a state under electrical stimulation, food or beverage to be consumed in a state under electrical stimulation, whose salty taste at the time of consuming is more effectively enhanced, and the like.

### Solution to Problem

The present inventors have conducted intensive studies in an attempt to solve the above-mentioned problems and found an effectively-enhanced salty taste when they consumed miso soup containing L-serine, while wearing an anode placed on the chin and a cathode on the posterior neck. Based on this finding, they have conducted further studies and completed the present invention.

Accordingly, the present invention provides the following.
[1] An enhancer of a salty taste of a food or beverage, that is consumed in a state under electrical stimulation, at the time of consuming the food or beverage, the enhancer comprising a hydroxyl group-containing amino acid or basic amino acid, or a salt thereof.
[2] The enhancer of the above-mentioned [1], wherein the electrical stimulation is that achieved by applying current to electrodes placed at two or more locations on the head and neck.
[3] The enhancer of the above-mentioned [2], wherein the two or more locations on the head and neck are the chin and the posterior neck.
[4] The enhancer of the above-mentioned [3], wherein the electrode placed on the chin is an anode and the electrode placed on the posterior neck is a cathode.
[5] The enhancer of any of the above-mentioned [1] to [4], wherein the state under electrical stimulation is a state in which the salty taste is enhanced by the electrical stimulation.
[6] The enhancer of any of the above-mentioned [1] to [5], further comprising an organic acid.
[7] The enhancer of any of the above-mentioned [1] to [6], wherein the food or beverage is a low-sodium food or beverage.
[8] The enhancer of any of the above-mentioned [1] to [7], wherein the hydroxyl group-containing amino acid or basic amino acid is L-serine.
[9] A food or beverage comprising a hydroxyl group-containing amino acid or basic amino acid, or a salt thereof, which is consumed in a state under electrical stimulation.
[10] The food or beverage of the above-mentioned [9], wherein the electrical stimulation is that achieved by applying current to electrodes placed at two or more locations on the head and neck.
[11] The food or beverage of the above-mentioned [10], wherein the two or more locations on the head and neck are the chin and the posterior neck.
[12] The food or beverage of the above-mentioned [11], wherein the electrode placed on the chin is an anode and the electrode placed on the posterior neck is a cathode.
[13] The food or beverage of any of the above-mentioned [9] to [12], wherein the state under electrical stimulation is a state in which the salty taste is enhanced by the electrical stimulation.
[14] The food or beverage of any of the above-mentioned [9] to [13], further comprising an organic acid.
[15] The food or beverage of any of the above-mentioned [9] to [14], wherein the food or beverage is a low-sodium food or beverage.
[16] The food or beverage of any of the above-mentioned [9] to [15], wherein the hydroxyl group-containing amino acid or basic amino acid is L-serine.
[17] A method for producing a food or beverage that is consumed in a state under electrical stimulation, comprising a step of adding a hydroxyl group-containing amino acid or basic amino acid, or a salt thereof.
[18] The production method of the above-mentioned [17], wherein the electrical stimulation is that achieved by applying current to electrodes placed at two or more locations on the head and neck.
[19] The production method of the above-mentioned [18], wherein the two or more locations on the head and neck are the chin and the posterior neck.
[20] The production method of the above-mentioned [19], wherein the electrode placed on the chin is an anode and the electrode placed on the posterior neck is a cathode.
[21] The production method of any of the above-mentioned [17] to [20], wherein the state under electrical stimulation is a state in which the salty taste is enhanced by the electrical stimulation.
[22] The production method of any of the above-mentioned [17] to [21], further comprising a step of adding an organic acid.
[23] The production method of any of the above-mentioned [17] to [22], wherein the food or beverage is a low-sodium food or beverage.
[24] The production method of any of the above-mentioned [17] to [23], wherein the hydroxyl group-containing amino acid or basic amino acid is L-serine.
[25] A method for enhancing a salty taste of a food or beverage, that is consumed in a state under electrical stimulation, at the time of consuming the food or beverage, the method comprising adding a hydroxyl group-containing amino acid or basic amino acid, or a salt thereof to the food or beverage.
[26] The enhancement method of the above-mentioned [25], wherein the electrical stimulation is that achieved by applying current to electrodes placed at two or more locations on the head and neck.
[27] The enhancement method of the above-mentioned [26], wherein the two or more locations on the head and neck are the chin and the posterior neck.
[28] The enhancement method of the above-mentioned [27], wherein the electrode placed on the chin is an anode and the electrode placed on the posterior neck is a cathode.
[29] The enhancement method of any of the above-mentioned [25] to [28], wherein the state under electrical stimulation is a state in which the salty taste is enhanced by the electrical stimulation.
[30] The enhancement method of any of the above-mentioned [25] to [29], further comprising adding an organic acid.
[31] The enhancement method of any of the above-mentioned [25] to [30], wherein the food or beverage is a low-sodium food or beverage.
[32] The enhancement method of any of the above-mentioned [25] to [31], wherein the hydroxyl group-containing amino acid or basic amino acid is L-serine.
[33] A method for enhancing a salty taste of a food or beverage at the time of consuming the food or beverage, comprising adding a hydroxyl group-containing amino acid or basic amino acid, or a salt thereof to the food or beverage, and applying, at the time of consuming the food or beverage, electrical stimulation to a person consuming the food or beverage.
[34] The enhancement method of the above-mentioned [33], wherein the electrical stimulation is that achieved by applying current to electrodes placed at two or more locations on the head and neck.
[35] The enhancement method of the above-mentioned [34], wherein the two or more locations on the head and neck are the chin and the posterior neck.
[36] The enhancement method of the above-mentioned [35], wherein the electrode placed on the chin is an anode and the electrode placed on the posterior neck is a cathode.
[37] The enhancement method of any of the above-mentioned [33] to [36], further comprising adding an organic acid to the food or beverage.
[38] The enhancement method of any of the above-mentioned [33]to [37], wherein the food or beverage is a low-sodium food or beverage.
[39] The enhancement method of any of the above-mentioned [33] to [38], wherein the hydroxyl group-containing amino acid or basic amino acid is L-serine.

### Advantageous Effects of Invention

The present invention provides an enhancer of a salty taste of food or beverage, that is consumed in a state under electrical stimulation, at the time of consuming the food or beverage, food or beverage to be consumed in a state under electrical stimulation, that can be consumed as having an enhanced salty taste, and the like.

### DESCRIPTION OF EMBODIMENTS

### 1. Enhancer of Salty Taste at the Time of Consuming Food or Beverage

The present invention provides an enhancer of a salty taste of a food or beverage, that is consumed in a state under electrical stimulation, at the time of consuming the food or beverage, the enhancer containing a hydroxyl group-containing amino acid or basic amino acid, or a salt thereof (hereinafter also to be referred to as “the enhancer of the present invention”).

Examples of the hydroxyl group-containing amino acid include serine, threonine, and the like. Among these, L-serine and L-threonine are preferred, and L-serine is more preferred.

Examples of the basic amino acid include lysine, arginine, histidine, and the like. Among these, L-lysine, L-arginine, and L-histidine are preferred, and L-lysine is more preferred.

Examples of the salt of hydroxyl group-containing amino acid include metal salts, ammonium salts, organic amine addition salts, amino acid addition salts, and the like.

Examples of the metal salt include alkali metal salts such as sodium salt, potassium salt, and the like, alkaline earth metal salts such as magnesium salt, calcium salt, and the like, aluminum salt, zinc salt, and the like. Examples of the ammonium salt include salts such as ammonium salt, tetramethylammonium salt, and the like. Examples of the organic amine addition salt include addition salts of morpholine, piperidine, or the like. Examples of the amino acid addition salt include addition salts of glycine, phenylalanine, asparagine, glutamine, or the like.

Examples of the salt of basic amino acid include acid addition salts, amino acid addition salts, and the like.

Examples of the acid addition salt include inorganic acid salts such as hydrochloride, sulfate, hydrobromide, nitrate, phosphate, and the like, and organic acid salts such as acetate, mesylate, succinate, maleate, fumarate, citrate, tartrate, and the like. Examples of the amino acid addition salt include addition salts of aspartic acid, glutamic acid, or the like.

When obtaining a salt of a hydroxyl group-containing amino acid or a basic amino acid, and the hydroxyl group-containing amino acid or basic amino acid is obtained in the form of a salt, it can be purified as is. When it is obtained in a free form, the hydroxyl group-containing amino acid or basic amino acid can be dissolved or suspended in a suitable solvent, and then isolated and purified after adding an acid or base.

In addition, hydroxyl group-containing amino acids, basic amino acids, or salts thereof may also exist in the form of adducts with various solvents such as water and the like. In the present invention, hydroxyl group-containing amino acids, basic amino acids, or salts thereof, including adducts thereof, are referred to as hydroxyl group-containing amino acids, basic amino acids, or salts thereof.

Hydroxyl group-containing amino acids, basic amino acids, or salts thereof may be synthesized by methods known per se, and commercially available products can also be preferably used.

In the enhancer of the present invention, the content of the hydroxyl group-containing amino acid or basic amino acid, or a salt thereof is not particularly limited. It is preferably 0.01% to 90% by weight, more preferably 0.1% to 70% by weight, further preferably 1% to 50% by weight, as the hydroxyl group-containing amino acids or basic amino acids.

In the present specification, the “food or beverage” is not particularly limited as long as it is served for consumption, and is preferably food or beverage containing a component that imparts salty taste. Examples of the component that imparts salty taste include sodium salt, potassium salt, magnesium salt, chloride, and the like, and among these, sodium salt is preferred. More specifically, examples of the component that imparts salty taste include sodium chloride (table salt), salt substitutes (e.g., potassium chloride, monosodium glutamate, lysine hydrochloride, etc.), and the like, and among these, sodium chloride is preferred. Furthermore, from the viewpoint of more effectively enhancing the salty taste at the time of consuming, the “food or beverage” is preferably “low-sodium food or beverage”. Furthermore, the food or beverage may also be health functional foods, foods for specified health uses, nutritional functional foods, dietary supplements, nutritional supplements, health supplements, medical foods, or the like.

In the present specification, the “low-sodium food or beverage” refers to food or beverage in which the sodium content is reduced by 84 mg or more per 100 g (100 ml for liquid products) of food or beverage, or food or beverage in which the reduction rate of sodium chloride content or sodium content is 20% or more, compared to standard food or beverage. Food or beverage in which the sodium content is reduced by 120 mg or more per 100 g (100 ml for liquid products) of food or beverage, or food or beverage in which the reduction rate of sodium chloride content or sodium content is 50% or more, compared to standard food or beverage, is more preferred, because the effect of adding the enhancer of the present invention can be remarkably demonstrated, i.e., a greater salt reduction effect can be expected. In addition, the amount of sodium chloride in low-sodium food or beverage at the time of consuming is preferably 0.1% to 0.7%, more preferably 0.2% to 0.5%.

In the present specification, “salty taste at the time of consuming food or beverage” means the salty taste perceived at the time of consuming food or beverage by a person consuming the food or beverage.

In the present specification, “food or beverage that is consumed in a state under electrical stimulation” (hereinafter also to be referred to as “electrical stimulation food or beverage”) refers to food or beverage prepared on the premise of applying electrical stimulation, for the purpose of making a person consuming same feel that the salty taste is enhanced at the time of consuming while applying electrical stimulation, or the like. It is preferably food or beverage prepared for the purpose of making a person consuming same feel that the salty taste is enhanced at the time of consuming while applying electrical stimulation, namely, food or beverage that is consumed in a state in which the salty taste is enhanced by the electrical stimulation.

The “electrical stimulation” is not particularly limited as long as the above-mentioned purpose is achieved. For example, electrical stimulations described in JP 2018-42991 A; JP 2017-163916 A; WO 2022/239857; TVRSJ, 2015, Vol. 20, No3, p. 239-242; Frontiers in Virtual Reality, 2022, Vol. 3, p. 1-10; Journal of Robotics and Mechatronics, 2021, 33(5), p. 1128-1134; “Entertainment Computing Symposium (EC2022)” September 2022, pp. 199-202, and the like can be mentioned.

More specifically, examples include electrical stimulation by applying current to electrodes placed at two or more locations on the head and neck. The two locations on the head and neck can be any part of the head and neck, and examples include the anterior neck, lateral neck, posterior neck, chin, mouth, cheek, parotid masseter muscle, occipital region, and the like. Among these, the anterior neck, posterior neck, chin, mouth, cheek, and occipital region are preferred, and among these, the posterior neck, chin, mouth, and occipital region are more preferred.

The electrodes placed at two or more locations on the head and neck refer to electrodes in a state where they can conduct electricity, consisting of at least one anode and at least one cathode in combination. In particular, a combination of an anode placed on the anterior neck, chin, mouth, or cheek and a cathode placed on the posterior neck or occipital region is preferred, and among these, a combination of an anode placed on the chin or mouth and a cathode placed on the posterior neck or occipital region is more preferred.

The conditions for conducting electricity are not particularly limited as long as the effects of the present invention are afforded. A direct current of 0.005 mA to 4.00 mA is preferred, a direct current of 0.01 mA to 3.80 mA is more preferred, a direct current of 0.10 mA to 3.50 mA is further preferred, a direct current of 1.00 mA to 3.30 mA is further more preferred, and a direct current of 2.00 mA to 3.00 mA is particularly preferred.

In addition, the enhancer of the present invention may contain an organic acid such as malic acid, lactic acid, or the like, or a salt thereof. When an organic acid or a salt thereof is contained in the enhancer of the present invention, the salty taste at the time of consuming food or beverage, with the enhancer of the present invention added thereto, is further enhanced.

Examples of the salt of organic acid include the salts exemplified above as salts of hydroxyl group-containing amino acid.

When obtaining a salt of an organic acid, and the organic acid is obtained in the form of a salt, it can be purified as is. When it is obtained in a free form, the organic acid can be dissolved or suspended in a suitable solvent, and then isolated and purified after adding an acid or base.

In addition, organic acids or salts thereof may also exist in the form of adducts with various solvents such as water and the like. In the present invention, organic acids or salts thereof, including adducts thereof, are referred to as organic acids or salts thereof.

Organic acids or salts thereof may be synthesized by methods known per se, and commercially available products can also be preferably used.

In the enhancer of the present invention, the content of the organic acid or a salt thereof is not particularly limited. It is preferably 0.001% to 30% by weight, more preferably 0.01% to 20% by weight, further preferably 0.1% to 10% by weight, as the organic acid.

Furthermore, the enhancer of the present invention may contain other components other than the hydroxyl group-containing amino acid or basic amino acid, or a salt thereof, to the extent that the effects of the present invention are not impaired. Other components can be appropriately selected depending on each form of the compositions described above. Examples include inorganic salts, acids, amino acids other than hydroxyl group-containing amino acids and basic amino acids, sugars, and various additives usable in food or beverage, such as seasonings, spices, colorants, color developers, emulsifiers, oils and fats, diluents, excipients, disintegrants, lubricants, binders, surfactants, water, physiological saline, vegetable oil solubilizers, isotonic agents, preservatives, antioxidants, and the like. Examples of the inorganic salt include potassium chloride, magnesium sulfate, and the like, examples of the acid include ascorbic acid, fumaric acid, malic acid, tartaric acid, citric acid, and salts thereof, examples of the amino acid other than hydroxyl group-containing amino acids and basic amino acids include glutamic acid and the like, and salts thereof, and examples of the sugar include sucrose, glucose, lactose, sugar, and the like. One or more kinds of other components may be contained as necessary.

The content of the above-mentioned other component(s) in the enhancer of the present invention is not particularly limited, and the total amount thereof in the enhancer of the present invention is preferably 0.000001% to 90% by weight, more preferably 0.00001% to 80% by weight, further preferably 0.00001% to 60% by weight, further more preferably 0.0001% to 40% by weight, and particularly preferably 0.0001% to 30% by weight.

The method for producing the enhancer of the present invention is now described.

The enhancer of the present invention can be produced by appropriately adding an organic acid or a salt thereof and/or the aforementioned other component(s) as necessary to the hydroxyl group-containing amino acid or basic amino acid, or a salt thereof, and mixing them as is, or by other production methods generally used in the field of food or beverage. The order of addition of these components is not particularly limited and can be set as appropriate according to the intended use.

Specific forms of the enhancer of the present invention include solid forms (tablets, chewable tablets, granules, powders, capsules, etc.), pastes, liquids, suspensions, and the like, and solid forms such as granules are preferred.

### 2. Food or Beverage Containing Hydroxyl Group-Containing Amino Acid or Basic Amino Acid, or Salt Thereof

The present invention also provides food or beverage containing a hydroxyl group-containing amino acid or basic amino acid, or a salt thereof, that is consumed in a state under electrical stimulation (hereinafter also referred to as “food or beverage of the present invention”).

The “hydroxyl group-containing amino acid or basic amino acid, or a salt thereof”, “food or beverage”, “electrical stimulation”, and “food or beverage that is consumed in a state under electrical stimulation” are as described above with respect to the enhancer of the present invention.

In the food or beverage of the present invention, the content of the hydroxyl group-containing amino acid or basic amino acid, or a salt thereof is not particularly limited. It is preferably 0.01% to 0.5% by weight, more preferably 0.03% to 0.4% by weight, further preferably 0.1% to 0.3% by weight.

In addition, the food or beverage of the present invention may contain an organic acid such as malic acid, lactic acid, or the like, or a salt thereof. When an organic acid or a salt thereof is contained in the food or beverage of the present invention, the salty taste at the time of consuming the food or beverage is further enhanced. The “organic acid or a salt thereof” is as described above with respect to the enhancer of the present invention.

In the food or beverage of the present invention, the content of the organic acid or a salt thereof is not particularly limited. It is preferably 0.001% to 0.05% by weight, more preferably 0.003% to 0.05% by weight, further preferably 0.01% to 0.04% by weight, as the organic acid.

Furthermore, the food or beverage of the present invention may contain other components other than the hydroxyl group-containing amino acid or basic amino acid, or a salt thereof, to the extent that the effects of the present invention are not impaired. Examples of the other component include inorganic salts, acids, amino acids other than hydroxyl group-containing amino acids and basic amino acids, sugars, and various additives usable in food or beverage, such as seasonings, spices, colorants, color developers, emulsifiers, oils and fats, diluents, excipients, disintegrants, lubricants, binders, surfactants, water, physiological saline, vegetable oil solubilizers, isotonic agents, preservatives, antioxidants, and the like. Examples of the inorganic salt include potassium chloride, magnesium sulfate, and the like, examples of the acid include ascorbic acid, fumaric acid, malic acid, tartaric acid, citric acid, and salts thereof, examples of the amino acid other than hydroxyl group-containing amino acids and basic amino acids include glutamic acid and the like, and salts thereof, and examples of the sugar include sucrose, glucose, lactose, sugar, and the like. One or more kinds of other components may be contained as necessary.

The content of the above-mentioned other component(s) in the food or beverage of the present invention is not particularly limited, and the total amount thereof in the food or beverage of the present invention is preferably 0.000001% to 30% by weight, more preferably 0.00001% to 20% by weight, further preferably 0.00001% to 10% by weight.

The method for producing the food or beverage of the present invention is now described.

The food or beverage of the present invention can be produced by appropriately adding an organic acid or a salt thereof and/or the aforementioned other component(s) as necessary to the raw materials or intermediate compositions before the final food or beverage, of the food or beverage, and mixing them as is, or by other production methods generally used in the field of food or beverage. The order of addition of these components is not particularly limited and can be set as appropriate according to the intended use.

### 3. Method for Producing Food or Beverage

The present invention also provides a method for producing a food or beverage that is consumed in a state under electrical stimulation, including a step of adding a hydroxyl group-containing amino acid or basic amino acid, or a salt thereof (hereinafter also to be referred to as “the production method of the present invention”).

The “hydroxyl group-containing amino acid or basic amino acid, or a salt thereof”, “electrical stimulation”, “food or beverage”, and “food or beverage that is consumed in a state under electrical stimulation” are as described above with respect to the enhancer of the present invention.

The amount of the hydroxyl group-containing amino acid or basic amino acid, or a salt thereof added to food or beverage that is consumed in a state under electrical stimulation is not particularly limited. It is preferably an amount such that the concentration of the hydroxyl group-containing amino acid or basic amino acid in the food or beverage becomes 0.01% to 0.5% by weight, more preferably 0.03% to 0.4% by weight, further preferably 0.1% to 0.3% by weight.

In addition, the production method of the present invention may further include a step of adding an organic acid such as malic acid, lactic acid, or the like, or a salt thereof, to food or beverage that is consumed in a state under electrical stimulation. The “organic acid or a salt thereof” is as described above with respect to the enhancer of the present invention.

The amount of the organic acid or a salt thereof added to food or beverage that is consumed in a state under electrical stimulation is not particularly limited. It is preferably an amount such that the concentration of the organic acid in the food or beverage becomes preferably 0.001% to 0.05% by weight, more preferably 0.003% to 0.05% by weight, further preferably 0.01% to 0.04% by weight.

In addition, the method of adding a hydroxyl group-containing amino acid or basic amino acid, or a salt thereof, or a hydroxyl group-containing amino acid or basic amino acid, or a salt thereof, and an organic acid or a salt thereof, to food or beverage that is consumed in a state under electrical stimulation is as described above for the method for producing the food or beverage of the present invention with respect to the food or beverage of the present invention.

By producing food or beverage that is consumed in a state under electrical stimulation by the production method of the present invention, food or beverage with enhanced salty taste at the time of consuming can be obtained.

### 4. Method 1 for Enhancing Salty Taste at the Time of Consuming Food or Beverage

The present invention also provides a method for enhancing a salty taste of a food or beverage, that is consumed in a state under electrical stimulation, at the time of consuming the food or beverage, characterized by adding a hydroxyl group-containing amino acid or basic amino acid, or a salt thereof to the food or beverage (hereinafter also to be referred to as “enhancement method 1 of the present invention”).

The “electrical stimulation”, “food or beverage that is consumed in a state under electrical stimulation”, “hydroxyl group-containing amino acid or basic amino acid, or a salt thereof”, “food or beverage”, and “salty taste at the time of consuming food or beverage” are as described above with respect to the enhancer of the present invention.

The amount of the hydroxyl group-containing amino acid or basic amino acid, or a salt thereof added to food or beverage that is consumed in a state under electrical stimulation is the same as the amount added of the hydroxyl group-containing amino acid or basic amino acid, or a salt thereof described above with respect to the production method of the present invention.

In addition, in the enhancement method 1 of the present invention, an organic acid such as malic acid, lactic acid, or the like, or a salt thereof, may be further added to food or beverage that is consumed in a state under electrical stimulation. By adding an organic acid or a salt thereof to the food or beverage, the salty taste at the time of consuming the food or beverage is further enhanced. The “organic acid or a salt thereof” is as described above with respect to the enhancer of the present invention.

The amount of the organic acid or a salt thereof added to food or beverage that is consumed in a state under electrical stimulation is the same as the amount added of the organic acid or a salt thereof described above with respect to the production method of the present invention.

In addition, the method of adding a hydroxyl group-containing amino acid or basic amino acid, or a salt thereof, or a hydroxyl group-containing amino acid or basic amino acid, or a salt thereof, and an organic acid or a salt thereof, to food or beverage that is consumed in a state under electrical stimulation is as described above for the method for producing the food or beverage of the present invention with respect to the food or beverage of the present invention.

### 5. Method 2 for Enhancing Salty Taste at the Time of Consuming Food or Beverage

The present invention also provides a method for enhancing a salty taste of a food or beverage at the time of consuming the food or beverage, characterized by adding a hydroxyl group-containing amino acid or basic amino acid, or a salt thereof to the food or beverage, and applying, at the time of consuming the food or beverage, electrical stimulation to a person consuming the food or beverage (hereinafter also to be referred to as “enhancement method 2 of the present invention”).

The “hydroxyl group-containing amino acid or basic amino acid, or a salt thereof”, “food or beverage”, and “electrical stimulation” are as described above with respect to the enhancer of the present invention. The amount of the hydroxyl group-containing amino acid or basic amino acid, or a salt thereof is the same as the amount added of the hydroxyl group-containing amino acid or basic amino acid, or a salt thereof described above with respect to the production method of the present invention.

The method of applying electrical stimulation is not particularly limited as long as the effects of the present invention are afforded. For example, the methods described in JP 2018-42991 A; JP 2017-163916 A; WO 2022/239857; TVRSJ, 2015, Vol. 20, No3, p. 239-242; Frontiers in Virtual Reality, 2022, Vol. 3, p. 1-10; Journal of Robotics and Mechatronics, 2021, 33(5), p. 1128-1134; “Entertainment Computing Symposium (EC2022)” September 2022, pp. 199-202, and the like can be mentioned.

More specifically, examples include a method of applying electrical stimulation by applying current to electrodes placed at two or more locations on the head and neck. The two locations on the head and neck can be any part of the head and neck, and examples include the anterior neck, lateral neck, posterior neck, chin, mouth, cheek, parotid masseter muscle, occipital region, and the like. Among these, the anterior neck, posterior neck, chin, mouth, cheek, and occipital region are preferred, and among these, the posterior neck, chin, mouth, and occipital region are more preferred.

The electrodes placed at two or more locations on the head and neck refer to electrodes in a state where they can conduct electricity, consisting of at least one anode and at least one cathode in combination. In particular, a combination of an anode placed on the anterior neck, chin, mouth, or cheek and a cathode placed on the posterior neck or occipital region is preferred, and among these, a combination of an anode placed on the chin or mouth and a cathode placed on the posterior neck or occipital region is more preferred.

The conditions for conducting electricity are not particularly limited as long as the effects of the present invention are afforded. A direct current of 0.005 mA to 4.00 mA is preferred, a direct current of 0.01 mA to 3.80 mA is more preferred, a direct current of 0.10 mA to 3.50 mA is further preferred, a direct current of 1.00 mA to 3.30 mA is further more preferred, and a direct current of 2.00 mA to 3.00 mA is particularly preferred.

In addition, in the enhancement method 2 of the present invention, an organic acid such as malic acid, lactic acid, or the like, or a salt thereof, may be further added to food or beverage. By adding an organic acid or a salt thereof to the food or beverage, the salty taste at the time of consuming the food or beverage is further enhanced. The “organic acid or a salt thereof” is as described above with respect to the enhancer of the present invention. The amount added of the organic acid or a salt thereof is the same as the amount added of the organic acid or a salt thereof described above with respect to the production method of the present invention.

In addition, the method of adding a hydroxyl group-containing amino acid or basic amino acid, or a salt thereof, or a hydroxyl group-containing amino acid or basic amino acid, or a salt thereof, and an organic acid or a salt thereof, to food or beverage is as described above for the method for producing the food or beverage of the present invention with respect to the food or beverage of the present invention.

The present invention is now specifically described with reference to Examples and the like, but the present invention is not limited in any way by these Examples. In the present specification, “%” means “% by weight” unless otherwise specified.

### EXAMPLE

In the Examples and the like, unless otherwise specified in each Example and the like, the following were used as raw materials.
L-serine: “L-serine” (manufactured by AJINOMOTO CO., INC.)
L-isoleucine: “L-isoleucine” (manufactured by AJINOMOTO CO., INC.)
L-glutamic acid: “L-glutamic acid” (manufactured by AJINOMOTO CO., INC.)
L-lysine: “L-lysine hydrochloride” (manufactured by AJINOMOTO CO., INC.)
glycine: “glycine” (manufactured by Organic Synthetic Pharmaceuticals Co., Ltd.)
malic acid: “Malic Acid Fuso” (FUSO CHEMICAL CO., LTD.)
lactic acid: “Fermented Lactic Acid 50% (PURAC KS50)” (Corbion Japan Co., Ltd.)
sodium chloride: “Naikai Salt” (Naikai Salt Industries Co., Ltd.)
Corn soup mix: “Knorr (registered trademark) Cup Soup Corn Cream” (manufactured by AJINOMOTO CO., INC.)

### Example 1 (Amino Acid Screening)

### (Preparation of Miso Soup)

Miso soup prepared using instant miso soup ingredients manufactured generally was adjusted to a salt concentration of 0.2% by adding sodium chloride. Then, L-serine, L-isoleucine, L-glutamic acid, L-lysine, or glycine (amino acid) was added to the miso soup to a concentration of 2.9 mmol/100 g to prepare each miso soup for evaluation. A miso soup without addition of amino acid was also prepared as a control.

### (Evaluation of Salty Taste)

The evaluation of salty taste was performed by a panel of four experts who are skilled in evaluation, in which they consumed each miso soup, prepared above, with or without electrical stimulation under the following conditions, then marked the evaluated salty taste intensity with a line on a scale representing evaluation criteria shown below, thus assigning a score to the salty taste intensity, and calculated the average of the scores obtained from the panel of four experts.

### [Electrical Stimulation Conditions]

electric current value: 2.00 mA, DC
electrode placement: anode: chin of each panel
   cathode: posterior neck of each panel

### [Evaluation Criteria]

0.00 points: no salty taste at all
1.00 points: tastes like miso soup prepared from instant miso soup mix and adjusted to salt concentration of 0.2% by adding sodium chloride
2.00 points: tastes like miso soup prepared from instant miso soup mix and adjusted to salt concentration of 0.4% by adding sodium chloride
3.00 points: tastes like miso soup prepared from instant miso soup mix and adjusted to salt concentration of 0.6% by adding sodium chloride

The evaluation results are shown in Table 1.

TABLE 1

| | No. | | | | | |
|---|---|---|---|---|---|---|
| | 1 | 2 | 3 | 4 | 5 | 6 |
| | no electrical stimulation | | | | | |
| amino acid | none | L-serine | L-isoleucine | L-glutamic | L-lysine | L-glycine |
| | | | | acid | | |
| average | 1.00 | 0.94 | 0.78 | 1.14 | 1.10 | 0.96 |
| evaluation | | | | | | |
| score | | | | | | |
| | No. | | | | | |
| | (1) | (2) | (3) | (4) | (5) | (6) |
| | with electrical stimulation | | | | | |
| amino acid | none | L-serine | L-isoleucine | L-glutamic | L-lysine | L-glycine |
| | | | | acid | | |
| average | 1.30 | 1.43 | 0.96 | 1.12 | 1.37 | 1.14 |
| evaluation | | | | | | |
| score | | | | | | |

As shown in Table 1, when L-serine or L-lysine was added to miso soup and electrical stimulation was applied at the time of consuming the miso soup to the person consuming the miso soup, the salty taste perceived by the person consuming the miso soup was enhanced compared to when L-serine or L-lysine was added but no electrical stimulation was applied, or when L-serine or L-lysine was not added but electrical stimulation was applied. In contrast, when amino acids other than L-serine and L-lysine were added, no enhancement in the salty taste perceived by the person consuming the miso soup was observed compared to when no amino acids were added but electrical stimulation was applied.

### Example 2 (Effect of Malic Acid Addition)

### (Preparation of Saline Solution)

A saline solution was prepared by adding sodium chloride to distilled water to adjust the salt concentration to 0.2%. Then, L-serine was added to the saline solution to a concentration of 0.30%, and/or malic acid was added to a concentration of 0.020%, to prepare each saline solution for evaluation. In addition, a 0.2% saline solution without addition of either L-serine or malic acid was prepared as a control.

### (Evaluation of Salty Taste)

The evaluation of salty taste was performed in the same manner as the evaluation of salty taste in Example 1, except that the number of expert panel members was three and the evaluation criteria were as shown below.

### [Evaluation Criteria]

0.00 points: no salty taste at all
1.00 points: salty taste of 0.2% saline solution was present
2.00 points: salty taste of 0.4% saline solution was present
3.00 points: salty taste of 0.6% saline solution was present

The evaluation results are shown in Table 2.

TABLE 2

| No. | 1 | 2 | 3 | 4 | (1) | (2) | (3) | (4) |
|---|---|---|---|---|---|---|---|---|
| electrical stimulation | − | − | − | − | + | + | + | + |
| L-serine addition | − | − | + | + | − | − | + | + |
| malic acid addition | − | + | − | + | − | + | − | + |
| average evaluation score | 1.00 | 0.97 | 1.14 | 1.17 | 1.36 | 1.26 | 1.49 | 1.70 |

As shown in Table 2, when malic acid was further added to the saline solution in addition to addition of L-serine to the saline solution and applying electrical stimulation at the time of consuming the saline solution to the person consuming the saline solution, a further enhanced salty taste was perceived by the person consuming the saline solution compared to when malic acid was not added.

### Example 3 (Effect of Lactic Acid Addition)

### (Preparation of Saline Solution)

A saline solution was prepared by adding sodium chloride to distilled water to adjust the salt concentration to 0.2%. Then, L-serine was added to the saline solution to a concentration of 0.30%, and lactic acid was added to a concentration of 0.02%, to prepare each saline solution for evaluation. In addition, a 0.2% saline solution without addition of lactic acid was prepared as a control.

### (Evaluation of Salty Taste)

The evaluation of salty taste was performed in the same manner as the evaluation of salty taste in Example 2.

The evaluation results are shown in Table 3.

TABLE 3

| No. | 1 | 2 |
|---|---|---|
| electrical stimulation + L-serine addition | − | − |
| lactic acid addition | − | + |
| average evaluation score | 1.00 | 1.11 |
| No. | (1) | (2) |
| electrical stimulation + L-serine addition | + | + |
| lactic acid addition | − | + |
| average evaluation score | 1.36 | 1.65 |

As shown in Table 3, when lactic acid was further added to the saline solution in addition to addition of L-serine to the saline solution and applying electrical stimulation at the time of consuming the saline solution to the person consuming the saline solution, a further enhanced salty taste was perceived by the person consuming the saline solution compared to when lactic acid was not added.

### Example 4 (Effect in Corn Soup)

Sodium chloride was added to corn soup prepared from corn soup mix to adjust the salt concentration to 0.2%. Then, L-serine was added to the corn soup to a concentration of 0.30%, and/or malic acid was added to the corn soup to a concentration of 0.020%, to prepare each corn soup for evaluation. In addition, a corn soup without addition of either L-serine or malic acid was prepared as a control.

### (Evaluation of Salty Taste)

The evaluation of salty taste was performed in the same manner as the evaluation of salty taste in Example 2.

The evaluation results are shown in Table 4.

TABLE 4

| No. | 1 | 2 | 3 | 4 | (1) | (2) | (3) | (4) |
|---|---|---|---|---|---|---|---|---|
| electrical stimulation | − | − | − | − | + | + | + | + |
| L-serine addition | − | − | + | + | − | − | + | + |
| malic acid addition | − | + | − | + | − | + | − | + |
| average evaluation score | 1.00 | 0.93 | 1.08 | 1.17 | 1.38 | 1.34 | 1.28 | 1.64 |

As shown in Table 4, when L-serine was added to corn soup and electrical stimulation was applied at the time of consuming the corn soup to the person consuming the corn soup, the salty taste perceived by the person consuming the corn soup was enhanced or tended to be enhanced compared to when L-serine was added but no electrical stimulation was applied, or when L-serine was not added but electrical stimulation was applied. In addition, when malic acid was further added to the corn soup in addition to addition of L-serine to the corn soup and applying electrical stimulation at the time of consuming the corn soup to the person consuming the corn soup, a further enhanced salty taste was perceived by the person consuming the corn soup compared to when malic acid was not added.

### INDUSTRIAL APPLICABILITY

The present invention can provide an enhancer of a salty taste of food or beverage, that is consumed in a state under electrical stimulation, at the time of consuming the food or beverage, food or beverage to be consumed in a state under electrical stimulation, that can be consumed as having an enhanced salty taste, and the like. Therefore, the present invention is useful in the field of food industry.

1. A food or beverage comprising L-serine or a salt thereof, which is consumed in a state under electrical stimulation.

2. The food or beverage according to claim 1, further comprising an organic acid.

3. The food or beverage according to claim 1, wherein the food or beverage is a low-sodium food or beverage.

4. A method for producing a food or beverage that is consumed in a state under electrical stimulation, comprising a step of adding a hydroxyl group-containing amino acid or basic amino acid, or a salt thereof.

5. The production method according to claim 4, wherein the hydroxyl group-containing amino acid or basic amino acid is L-serine.

6. The production method according to claim 5, further comprising a step of adding an organic acid.

7. The production method according to claim 5, wherein the food or beverage is a low-sodium food or beverage.

8. A method for enhancing a salty taste of a food or beverage, that is consumed in a state under electrical stimulation, at the time of consuming the food or beverage, the method comprising adding a hydroxyl group-containing amino acid or basic amino acid, or a salt thereof to the food or beverage.

9. The enhancement method according to claim 8, wherein the electrical stimulation is that achieved by applying current to electrodes placed at two or more locations on the head and neck.

10. The enhancement method according to claim 9, wherein the two or more locations on the head and neck are the chin and the posterior neck, and the electrode placed on the chin is an anode and the electrode placed on the posterior neck is a cathode.

11. The enhancement method according to claim 8, wherein the state under electrical stimulation is a state in which the salty taste is enhanced by the electrical stimulation.

12. The enhancement method according to claim 8, further comprising adding an organic acid.

13. The enhancement method according to claim 8, wherein the food or beverage is a low-sodium food or beverage.

14. The enhancement method according to claim 8, wherein the hydroxyl group-containing amino acid or basic amino acid is L-serine.

15. A method for enhancing a salty taste of a food or beverage at the time of consuming the food or beverage, comprising adding a hydroxyl group-containing amino acid or basic amino acid, or a salt thereof to the food or beverage, and applying, at the time of consuming the food or beverage, electrical stimulation to a person consuming the food or beverage.

16. The enhancement method according to claim 15, wherein the electrical stimulation is that achieved by applying current to electrodes placed at two or more locations on the head and neck.

17. The enhancement method according to claim 16, wherein the two or more locations on the head and neck are the chin and the posterior neck, and the electrode placed on the chin is an anode and the electrode placed on the posterior neck is a cathode.

18. The enhancement method according to claim 15, further comprising adding an organic acid to the food or beverage.

19. The enhancement method according to claim 15, wherein the food or beverage is a low-sodium food or beverage.

20. The enhancement method according to claim 15, wherein the hydroxyl group-containing amino acid or basic amino acid is L-serine.
